(19) United States
(12) Patent Application Publication
(10) Pub. No.: US 2020/0327689 A1
(43) Pub. Date: Oct. 15, 2020

(54) DYNAMIC IMAGE ANALYSIS APPARATUS, DYNAMIC IMAGE ANALYSIS SYSTEM AND STORAGE MEDIUM

(71) Applicant: Konica Minolta, Inc., Tokyo (JP)

(72) Inventors: Akinori TSUNOMORI, Tokyo (JP); Ryoichi Watanabe, Toyohashi-shi (JP); Shinsuke Katsuhara, Tokyo (JP)

(21) Appl. No.: 16/812,689

(22) Filed: Mar. 9, 2020

(30) Foreign Application Priority Data
Apr. 9, 2019 (JP) 2019-074282

(51) Int. Cl. G06T 7/62; G06T 7/00; G16H 30/20

(52) U.S. Cl. CPC G06T 7/62; G06T 7/0012; G06T 2207/30061; G06T 2207/10116; G16H 30/20

(57) ABSTRACT

A dynamic image analysis apparatus includes a hardware processor. An imaging condition affects the area and the volume of a subject that are calculated from dynamic images obtained by radiographing a cyclic dynamic state of the subject from different directions. Based on a set value of the imaging condition in the radiographing, the hardware processor corrects the area or the volume of the subject calculated from the dynamic images. Further, based on (i) the volume calculated based on the corrected area or (ii) the corrected volume, the hardware processor estimates an evaluation index of a function of the subject.

### CROSS-REFERENCE TO RELATED APPLICATIONS

The entire disclosure of Japanese Patent Application No. 2019-074282 filed on Apr. 9, 2019 is incorporated herein by reference in its entirety.

### BACKGROUND

### Technological Field

The present disclosure relates to a dynamic image analysis apparatus, a dynamic image analysis system and a storage medium.

### Description of the Related Art

As disclosed, for example, in Non-Patent Document 1 (P. C. Pratt et al., “A Method for the Determination of Total Lung Capacity from Posteroanterior and Lateral Chest Roentgenograms”, American Review of Respiratory Disease, 96(3), pp. 548-552, 1967), there has been developed a method for estimating TLC (Total Lung Capacity) from the areas of lung fields in a plain X-ray image of the front of a chest and a plain X-ray image of a side of the chest that have been taken independently.

### SUMMARY

There is disclosed in Non-Patent Document 1 that the volume of the lung fields is estimated on the basis of the areas of the lung fields calculated from each of the images at the forced maximal inspiratory position obtained by radiographing the front and the side of the chest independently, and TLC is estimated on the basis of the estimated volume. However, it often happens that timings regarded as the forced maximal inspiratory position in these two times of imaging deviate, and accordingly the lung fields in the images do not match in size. As a result, the volume of the lung fields cannot be accurately estimated, and accordingly TLC cannot be accurately estimated either. Further, even when the same lung fields are imaged, the areas of the lung fields that are calculated from the image differ depending on an imaging condition(s). Non-Patent Document 1 does not take this point into account in particular. Still further, the method disclosed in Non-Patent Document 1 is for estimating TLC only, and cannot be used for obtaining other indexes important in evaluating a respiratory function (respiratory function indexes), such as RV (Residual Volume), and a lung volume curve.

Objects of the present invention include (i) by using dynamic images, improving estimation accuracy of the volume of a subject that moves and improving estimation accuracy of evaluation indexes for evaluating functions of the subject, the evaluation indexes being estimated on the basis of the volume of the subject, and (ii) obtaining evaluation indexes for evaluating the functions of the subject, the evaluation indexes being unobtainable from plain X-ray images.

In order to achieve at least one of the objects, according to a first aspect of the present invention, there is provided a dynamic image analysis apparatus including a hardware processor that:

from each frame image of each of dynamic images obtained by radiographing a cyclic dynamic state of a subject from different directions, calculates a characteristic amount relating to the dynamic state of the subject;

based on the calculated characteristic amount, extracts at least one set of frame images having phases of the dynamic state of the subject most similar to one another from the dynamic images;

from each of the frame images of each of the extracted at least one set, calculates an area of the subject;

based on the calculated area of the subject, calculates a volume of the subject for each of the extracted at least one set;

based on a set value of an imaging condition in the radiographing, the imaging condition affecting the area and the volume of the subject that are calculated from the dynamic images, corrects (i) the calculated area of the subject or (ii) the calculated volume of the subject; and

based on (i) the volume calculated based on the corrected area or (ii) the corrected volume, estimates an evaluation index of a function of the subject.

According to a second aspect of the present invention, there is provided a dynamic image analysis system including:

the dynamic image analysis apparatus;

an imaging apparatus that radiographs the dynamic state of the subject; and

a display apparatus that displays the estimated evaluation index.

According to a third aspect of the present invention, there is provided a non-transitory computer-readable storage medium storing a program to cause a computer to:

from each frame image of each of dynamic images obtained by radiographing a cyclic dynamic state of a subject from different directions, calculate a characteristic amount relating to the dynamic state of the subject;

based on the calculated characteristic amount, extract at least one set of frame images having phases of the dynamic state of the subject most similar to one another from the dynamic images;

from each of the frame images of each of the extracted at least one set, calculate an area of the subject;

based on the calculated area of the subject, calculate a volume of the subject for each of the extracted at least one set;

based on a set value of an imaging condition in the radiographing, the imaging condition affecting the area and the volume of the subject that are calculated from the dynamic images, correct (i) the calculated area of the subject or (ii) the calculated volume of the subject; and

based on (i) the volume calculated based on the corrected area or (ii) the corrected volume, estimate an evaluation index of a function of the subject.

### BRIEF DESCRIPTION OF THE DRAWINGS

The objects, advantages, and characteristics provided by one or more embodiments of the present invention will become more fully understood from the detailed description given hereinbelow and the appended drawings that are given by way of illustration only, and thus are not intended as a definition of the limits of the present invention, wherein:

FIG. 1 shows an overall configuration of a dynamic image analysis system according to an embodiment(s);

FIG. 2 is a flowchart showing an imaging control process that is performed by a controller of an imaging console shown in FIG. 1;

FIG. 3 is a flowchart showing a respiratory function index estimation process that is performed by a controller of a diagnostic console shown in FIG. 1;

FIG. 4A shows the lower ends of lung fields in a frontal chest radiograph;

FIG. 4B shows the lower ends of the lung fields in a lateral chest radiograph;

FIG. 5A is an illustration to explain a method for identifying the lower end of a lung field in a lateral chest radiograph;

FIG. 5B is an illustration to explain another method for identifying the lower end of the lung field in the lateral chest radiograph;

FIG. 5C is an illustration to explain another method for identifying the lower end of the lung field in the lateral chest radiograph;

FIG. 6A is an illustration to explain a characteristic amount calculated from a frame image of a frontal chest dynamic image;

FIG. 6B is an illustration to explain the characteristic amount calculated from a frame image of a lateral chest dynamic image;

FIG. 7 shows an example of a graph where temporal change of a lung-apex-to-diaphragm distance in each of the frontal chest dynamic image and the lateral chest dynamic image is plotted;

FIG. 8 is an illustration to explain a method for calculating the width of shoulders;

FIG. 9 is a diaphragm to explain a method for generating a lung volume curve;

FIG. 10 is a diagram to explain a method for estimating respiratory function indexes;

FIG. 11 is a diagram to explain an algorithm for automatically determining a respiratory state during dynamic imaging;

FIG. 12 shows an example of how estimation results of respiratory function indexes are displayed;

FIG. 13 shows another example of how the estimation results of the respiratory function indexes are displayed;

FIG. 14 shows another example of how the estimation results of the respiratory function indexes are displayed;

FIG. 15 shows another example of how the estimation results of the respiratory function indexes are displayed;

FIG. 16 shows another example of how the estimation results of the respiratory function indexes are displayed;

FIG. 17 shows another example of how the estimation results of the respiratory function indexes are displayed;

FIG. 18 shows another example of how the estimation results of the respiratory function indexes are displayed; and

FIG. 19 shows another example of how the estimation results of the respiratory function indexes are displayed.

### DETAILED DESCRIPTION OF EMBODIMENTS

Hereinafter, one or more embodiments of the present invention are described in detail with reference to the drawings. However, the scope of the present invention is not limited to the embodiments or illustrated examples.

### [Configuration of Dynamic Image Analysis System 100]

First, configuration of a dynamic image analysis system according to an embodiment(s) will be described.

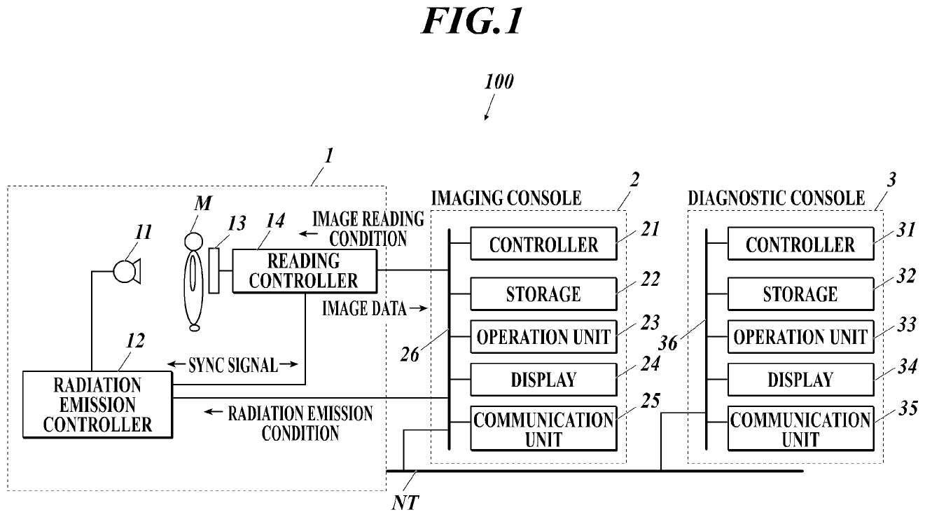

FIG. 1 shows an overall configuration of a dynamic image analysis system 100 according to an embodiment(s).

As shown in FIG. 1, the dynamic image analysis system 100 includes: an imaging apparatus 1; an imaging console 2 connected with the imaging apparatus 1 via a communication cable or the like; and a diagnostic console 3 connected with the imaging console 2 via a communication network NT, such as a LAN (Local Area Network). The apparatuses or the like of the dynamic image analysis system 100 are in conformity with DICOM (Digital Image and Communications in Medicine) standard and communicate with one another in conformity with DICOM.

### [Configuration of Imaging Apparatus 1]

The imaging apparatus 1 is an imager that images/photographs a cyclic dynamic state of a chest. Examples of the cyclic dynamic state include: change in shape of the lungs, namely expansion and contraction of the lungs, with respiration; and pulsation of the heart. Dynamic imaging is performed by repeatedly emitting pulsed radiation, such as X-rays, to a subject at predetermined time intervals (pulse emission) or continuously emitting radiation without a break to the subject at a low dose rate (continuous emission), thereby generating a plurality of images showing the dynamic state of the subject. A series of images obtained by dynamic imaging is called a dynamic image. Images constituting a dynamic image are called frame images. In the embodiment(s) described below, dynamic imaging of the front of a chest and dynamic imaging of a side of the chest are performed by pulse emission as an example.

A radiation source 11 is arranged so as to face a radiation detector 13 with the subject in between, and emits radiation (X-rays) to the subject under the control of a radiation emission controller 12.

The radiation emission controller 12 is connected with the imaging console 2, and controls the radiation source 11 on the basis of radiation emission conditions input from the imaging console 2 to perform radiographing. The radiation emission conditions input from the imaging console 2 include a pulse rate, a pulse width, a pulse interval, the number of frame images to be taken by one dynamic imaging, a value of current of an X-ray tube, a value of voltage of the X-ray tube, a type of added filter and SID (the shortest distance between the tube of the radiation source 11 and the radiation detector 13). The pulse rate is the number of times radiation is emitted per second, and matches the frame rate described below. The pulse width is a period of time for one radiation emission. The pulse interval is a period of time from start of one radiation emission to start of the next radiation emission, and matches the frame interval described below.

The radiation detector 13 is constituted of a semiconductor image sensor, such as an FPD (Flat Panel Detector). The FPD is constituted of detection elements (pixels) arranged at predetermined points on a substrate, such as a glass substrate, in a matrix. The detection elements detect radiation (intensity of radiation) that has been emitted from the radiation source 11 and passed through at least the subject, convert the detected radiation into electric signals, and accumulate the electric signals therein. The pixels are provided with switching elements, such as TFTs (Thin Film Transistors). There are an indirect conversion FPD that converts X-rays into electric signals with photoelectric conversion element(s) via scintillator(s) and a direct conversion FPD that directly converts X-rays into electric signals. Either of these can be used.

The radiation detector 13 is arranged so as to face the radiation source 11 with the subject in between.

A reading controller 14 is connected with the imaging console 2. The reading controller 14 controls the switching elements of the pixels of the radiation detector 13 on the basis of image reading conditions input from the imaging console 2 to switch the pixels from which the electric signals accumulated in the pixels are read, thereby reading the electric signals accumulated in the radiation detector 13 and obtaining image data. The image data is constituted of signal values indicating density values of the pixels. This image data is a frame image(s). The reading controller 14 outputs the obtained frame images to the imaging console 2. The image reading conditions include a frame rate, a frame interval, a sampling pitch (pixel size) and an image size (matrix size). The frame rate is the number of frame images to be obtained per second, and matches the pulse rate described above. The frame interval is a period of time from start of one frame image obtaining to start of the next frame image obtaining, and matches the pulse interval described above.

The radiation emission controller 12 and the reading controller 14 are connected with one another, and exchange sync signals to synchronize radiation emission and image reading with one another.

### [Configuration of Imaging Console 2]

The imaging console 2 outputs the radiation emission conditions and the image reading conditions to the imaging apparatus 1 to control radiographing and radiograph reading that are performed by the imaging apparatus 1, and also displays dynamic images obtained (generated) by the imaging apparatus 1 so that a radiographer (user), such as a radiologist, can check if positioning has no problem, and also can determine if the dynamic images are suitable for diagnosis.

The imaging console 2 includes, as shown in FIG. 1, a controller 21, a storage 22, an operation unit 23, a display 24 and a communication unit 25. These components are connected with one another via a bus 26.

The controller 21 includes a CPU (Central Processing Unit) and a RAM (Random Access Memory). The CPU of the controller 21 reads a system program(s) and various process programs stored in the storage 22 in response to the radiographer operating the operation unit 23, loads the read programs into the RAM, and performs various processes, such as the below-described imaging control process, in accordance with the loaded programs, thereby performing concentrated control of operation of each component of the imaging console 2 and radiation emission and image reading of the imaging apparatus 1.

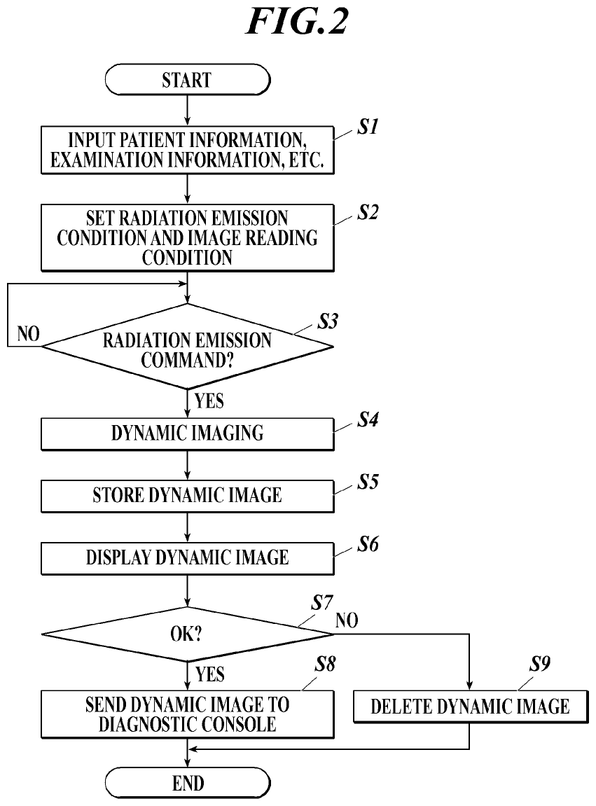

The storage 22 is constituted of a nonvolatile semiconductor memory, a hard disk and/or the like. The storage 22 stores various programs to be executed by the controller 21, parameters necessary to perform processes of the programs, data, such as process results, and so forth. For example, the storage 22 stores a program for the imaging control process shown in FIG. 2. The storage 22 also stores the radiation emission conditions and the image reading conditions associated with respective subjects (in this embodiment, chest). The programs are stored in the form of a computer readable program code(s), and the controller 21 operates in accordance with the program code.

The operation unit 23 includes: a keyboard including cursor keys, number input keys and various function keys; and a pointing device, such as a mouse, and outputs, to the controller 21, command signals input by the radiographer operating the keys of the keyboard or the mouse. The operation unit 23 may have a touchscreen on the display screen of the display 24. In this case, the operation unit 23 outputs command signals input via the touchscreen to the controller 21.

The display 24 is constituted of a monitor, such as an LCD (Liquid Crystal Display) or a CRT (Cathode Ray Tube), and displays commands input from the operation unit 23, data and so forth in accordance with commands of display signals input from the controller 21.

The communication unit 25 includes a LAN adapter, a modem and a TA (Terminal Adapter), and controls data exchange with apparatuses connected to the communication network NT.

### [Configuration of Diagnostic Console 3]

The diagnostic control 3 is a dynamic image analysis apparatus that obtains dynamic images from the imaging console 2, and displays the obtained dynamic images and/or analysis results of the dynamic images to help a doctor(s) make a diagnosis.

The diagnostic console 3 includes, as shown in FIG. 1, a controller 31 (hardware processor), a storage 32, an operation unit 33, a display 34 and a communication unit 35. These components are connected with one another via a bus 36.

The controller 31 includes a CPU and a RAM. The CPU of the controller 31 reads a system program(s) and various process programs stored in the storage 32 in response to a user operating the operation unit 33, loads the read programs into the RAM, and performs various processes, such as the below-described respiratory function index estimation process, in accordance with the loaded programs, thereby performing concentrated control of operation of each component of the diagnostic console 3.

The storage 32 is constituted of a nonvolatile semiconductor memory, a hard disk and/or the like. The storage 32 stores various programs, including a program for the respiratory function index estimation process, to be executed by the controller 31, parameters necessary to perform processes of the programs, data, such as process results, and so forth. The programs are stored in the form of a computer readable program code(s), and the controller 31 operates in accordance with the program code.

The storage 32 also stores the dynamic images associated with patient information (e.g. patient ID, name, height, weight, age, sex, etc.) and examination (test) information (e g examination (test) ID, examination (testing) date, subject (in this embodiment, chest), imaging direction (front, side), posture (standing, decubitus (supine)), lung field (left, right) closer to the radiation detector 13 in a case where the imaging direction is a side, respiratory state (quiet (resting) breathing, deep breathing, quiet breathing and deep breathing, breath holding, etc.), etc.).

The storage 32 also stores imaging conditions that affect the area and the volume that are calculated from images obtained by imaging (e.g. SID, distance between an examinee M (subject) and the radiation detector 13, etc.) associated with their reference values (e.g. BASE_{SID}, BASEₛᵤₚᵢₙₑ, etc. described below) and correction coefficients (e.g. α_{SID}, αₛᵤₚᵢₙₑ, etc. described below).

The storage 32 also stores known calculation formulae (detailed below) for calculating predictive values of respiratory function indexes of the examinee M (subject) from the height and/or age of the examinee M.

The operation unit 33 includes: a keyboard including cursor keys, number input keys and various function keys; and a pointing device, such as a mouse, and outputs, to the controller 31, command signals input by the user operating the keys of the keyboard or the mouse. The operation unit 33 may have a touchscreen on the display screen of the display 34. In this case, the operation unit 33 outputs command signals input via the touchscreen to the controller 31.

The display 34 is constituted of a monitor, such as an LCD or a CRT, and performs various types of display in accordance with commands of display signals input from the controller 31. The display 34 may be a display apparatus separate from the diagnostic console 3 (e.g. a display apparatus connected to the diagnostic console 3 via the communication unit 35).

The communication unit 35 includes a LAN adapter, a modem and a TA, and controls data exchange with apparatuses connected to the communication network NT.

### [Operation of Dynamic Image Analysis System 100]

Next, operation of the dynamic image analysis system 100 will be described.

### [Operation of Imaging Apparatus 1 and Imaging Console 2]

First, imaging that is performed by the imaging apparatus 1 and the imaging console 2 will be described.

FIG. 2 shows the imaging control process that is performed by the controller 21 of the imaging console 2. The imaging control process is performed by the controller 21 in cooperation with the program stored in the storage 22.

First, a radiographer operates the operation unit 23 of the imaging console 2 to input patient information on an examinee M and examination information (Step S1).

Next, the controller 21 reads radiation emission conditions from the storage 22 to set them in the radiation emission controller 12, and also reads image reading conditions from the storage 22 to set them in the reading controller 14 (Step S2).

Next, the controller 21 waits for a radiation emission command to be input by the radiographer operating the operation unit 23 (Step S3). The radiographer places a subject (of the examinee M) between the radiation source 11 and the radiation detector 13 and performs positioning. Also, the radiographer instructs the examinee M about the respiratory state (quiet breathing, deep breathing, quiet breathing and deep breathing, etc.). When preparations for imaging are complete, the radiographer operates the operation unit 23 to input a radiation emission command.

When receiving the radiation emission command input with the operation unit 23 (Step S3; YES), the controller 21 outputs an imaging start command to the radiation emission controller 12 and the reading controller 14 to start dynamic imaging (Step S4). In response to the imaging start command, the radiation source 11 emits radiation at pulse intervals set in the radiation emission controller 12, and accordingly the radiation detector 13 obtains (generates) a series of frame images. The radiographer may instruct the examinee M about the respiratory state while dynamic imaging is being performed.

When dynamic imaging for a predetermined number of frame images finishes, the controller 21 outputs an imaging end command to the radiation emission controller 12 and the reading controller 14 to stop dynamic imaging. The number of frame images to be taken covers at least one cycle of respiration.

The frame images obtained by dynamic imaging are successively input to the imaging console 2 and stored in the storage 22 associated with respective numbers (frame numbers) indicating what number in the imaging order the respective frame images have been taken (Step S5) and displayed on the display 24 (Step S6). The radiographer checks the positioning or the like with the displayed dynamic image, and determines whether the dynamic image obtained by dynamic imaging is suitable for diagnosis (Imaging OK) or re-imaging is necessary (Imaging NG). Then, the radiographer operates the operation unit 23 to input the determination result.

If the radiographer inputs the determination result “Imaging OK” by operating the operation unit 23 (Step S7; YES), the controller 21 attaches, to the respective frame images obtained by dynamic imaging (e.g. writes, in the header region of the image data in DICOM), an ID to identify the dynamic image, the patient information, the examination information, the radiation emission conditions, the image reading conditions, the respective numbers (frame numbers) indicating what number in the imaging order the respective frame images have been taken and other information, and sends the same to the diagnostic console 3 through the communication unit 25 (Step S8), and then ends the imaging control process. If the radiographer inputs the determination result “Imaging NG” by operating the operation unit 23 (Step S7; NO), the controller 21 deletes (the series of) the frame images from the storage 22 (Step S9), and then ends the imaging control process. In this case, re-imaging is necessary.

In this embodiment, after dynamic imaging of the front of the chest (or side of the chest) is performed by following the imaging control process, dynamic imaging of a side of the chest (or front of the chest) is performed thereby. As a result, a dynamic image of the front of the chest (hereinafter “frontal chest dynamic image”) and a dynamic image of the side of the chest (hereinafter “lateral chest dynamic image”) are obtained.

### [Operation of Diagnostic Console 3]

Next, operation of the diagnostic console 3 will be described.

In the diagnostic console 3, when receiving a series of frame images of a dynamic image from the imaging console 2 through the communication unit 35, the controller 31 stores the received dynamic image in the storage 32.

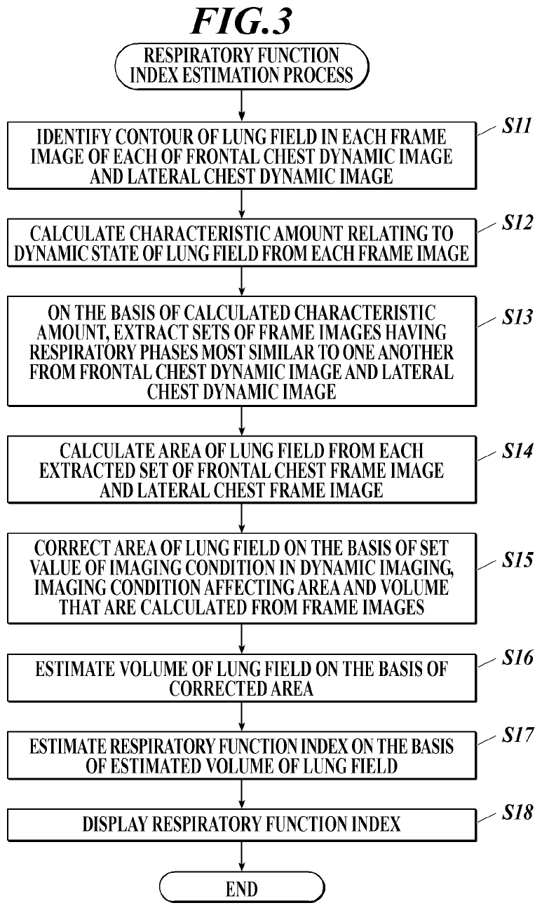

When a user selects, with the operation unit 33, a frontal chest dynamic image and a lateral chest dynamic image of the same patient from dynamic images stored in the storage 32, and instructs the diagnostic console 3 to estimate respiratory function indexes, the controller 31 performs the respiratory function index estimation process shown in FIG. 3 in cooperation with the program stored in the storage 32. The respiratory function indexes are indexes for evaluating a respiratory function of the lungs.

Hereinafter, the respiratory function index estimation process will be described with reference to FIG. 3.

In the respiratory function index estimation process, dynamic images each constituted of all frame images obtained by dynamic imaging may be used, or dynamic images each constituted of some of all the frame images obtained by dynamic imaging may be used.

First, the controller 31 reads the selected frontal chest dynamic image and lateral chest dynamic image from the storage 32, and identifies contours of the lung fields in each frame image of each dynamic image (Step S11).

The contours of the lung fields may be identified using any known method.

For example, the controller 31 may cause the display 34 to display each frame image, and identify the contours of the lung fields on the basis of contours (lines or points) specified on the displayed frame image by the user operating the operation unit 33. In this case, in order to improve identification accuracy of the contours of the lung fields, by using a method disclosed in Reference Document 1 (JP 5,814,655 B), the controller 31 may automatically correct the specified contours on the basis of a moving direction of a movable point on the contours with respect to a straight line that passes through the movable point and the centroid of the contours.

The contours of the lung fields may be automatically identified using known image processing, such as edge detection, dynamic contour model or region segmentation. Usable methods are disclosed, for example, in Reference Document 2 (JP 2004-188202 A) and Reference Document 3 (Francisco M. Carrascal et al., “Automatic calculation of total lung capacity from automatically traced lung boundaries in postero-anterior and lateral digital chest radiographs”, Med. Phys. VOL. 25, No. 7, pp. 1117-1131, July 1998).

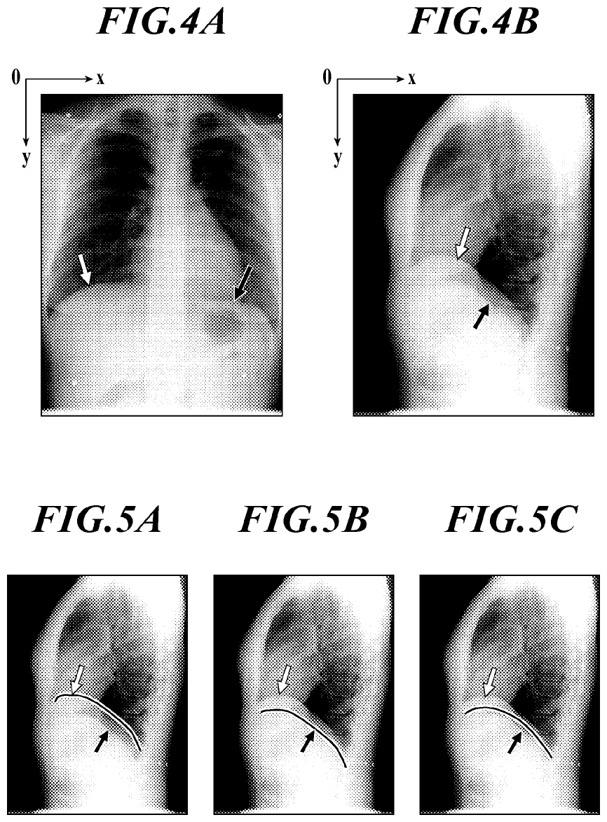

FIG. 4A shows an example of a frontal chest radiograph. FIG. 4B shows an example of a lateral chest radiograph. As indicated by arrows in FIG. 4A, the lower ends of the left lung and the right lung are different from one another in position, and as indicated by arrows in FIG. 4B, in the lateral chest radiograph, two edges exist as the lower ends of the lung fields (left and right lungs). In each frame image of the lateral chest dynamic image, the lower end of a lung field (hereinafter “lateral lung field”) as a lung field (lung field region) that includes both lungs can be identified using any of the following methods (1) to (3).

(1) Identify, between the two edges, the inner (upper) edge as the lower end of the lateral lung field (FIG. 5A).
(2) Identify, between the two edges, the outer (lower) edge as the lower end of the lateral lung field (FIG. 5B).
(3) Identify a representative value (e.g. the mean value) of the edge identified by (1) and the edge identified by (2) as the lower end of the lateral lung field (FIG. 5C).

The method (3) is preferable because it has a small error.

Next, the controller 31 calculates a characteristic amount relating to the dynamic state of the lung fields from each frame image of each of the frontal chest dynamic image and the lateral chest dynamic image (Step S12).

Respiration is constituted of expiratory phases and inspiratory phases. During the expiratory phases, the diaphragm rises, so that air is released from the lungs (lung fields), and accordingly the lung fields become small (contract). This increases density of the lung fields, and in a dynamic image, the lung fields are depicted in low density values (signal values). At the maximal expiratory position, the position of the diaphragm is the highest. During the inspiratory phases, the diaphragm lowers, so that air is taken into the lungs (lung fields), and accordingly the lung fields become large (expand). This decreases density of the lung fields, and in the dynamic image, the lung fields are depicted in high density values. At the maximal inspiratory position, the position of the diaphragm is the lowest. The density in the lung fields, the areas of the lung fields and the vertical position of the diaphragm (or a distance between the lung apex and the apex of the diaphragm (hereinafter “lung-apex-to-diaphragm distance”) or a distance between the aortic arch and the apex of the diaphragm because the lung apex and the aortic arch hardly move) are types of the characteristic amount relating to the dynamic state of the lung fields due to the respiration.

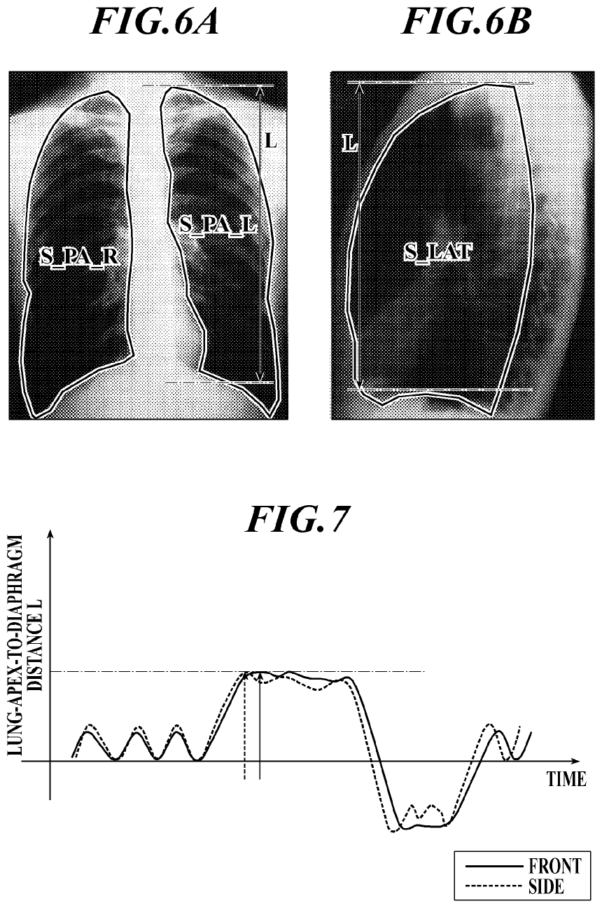

In Step S12, as shown in FIG. 6A and FIG. 6B, the controller 31 calculates, as the characteristic amount relating to the dynamic state of the lung fields, a lung-apex-to-diaphragm distance L in each frame image.

Next, on the basis of the calculated characteristic amount, the controller 31 extracts at least one set of frame images having the respiratory phases most similar to one another from the frontal chest dynamic image and the lateral chest dynamic image (Step S13).

FIG. 7 shows an example of a graph where temporal change of the lung-apex-to-diaphragm distance L in each of the frontal chest dynamic image and the lateral chest dynamic image is plotted. In Step S13, the controller 31 extracts, as each set of frame images having the most similar respiratory phases, for example, a set of frame images having the most similar values (smallest difference) of the lung-apex-to-diaphragm distance L from the frontal chest dynamic image and the lateral chest dynamic image as indicated by arrows in FIG. 7. In this embodiment, the controller 31 extracts multiple sets of frame images from the frontal chest dynamic image and the lateral chest dynamic image along the time axis, each set having the most similar values of the characteristic amount, which have been calculated in Step S12. These sets of frame images may be extracted at equal intervals or unequal intervals. When the frontal chest dynamic image and the lateral chest dynamic image have different frame rates, the controller 31 may extract the sets of frame images in accordance with the dynamic image having a lower frame rate.

Next, the controller 31 calculates the areas of the lung fields from each extracted set of frame images (hereinafter “frontal chest frame image” and “lateral chest frame image”) (Step S14).

In Step S14, as shown in FIG. 6A and FIG. 6B, the controller 31 calculates, for each extracted set, the area S_PA_R of the right lung in the frontal chest frame image, the area S PAL of the left lung in the frontal chest frame image, and the area S_LAT of the lateral lung field in the lateral chest frame image. The area S (S_PA_R, S_PA_L, S_LAT) of each lung field can be obtained by Equation 1 below on the basis of the number of pixels NoP inside the contours of each lung field and a sampling pitch SP.

S=NoP×SP×SP (Equation 1)

The area S_LAT of the lateral lung field may be obtained from each lateral chest frame image by the following (1) and (2) instead of taking the inside of the contours identified in Step S11 as the lateral lung field. This leads to estimating the volume of the lung fields by taking the sizes of the left lung and the right lung into account without distinguishing the left lung and the right lung from one another.

(1) Calculate the areas of the left and right lung fields from the lateral chest frame image without distinguishing the left lung and the right lung from one another. More specifically, calculate the area of a lung field having the upper edge in FIG. 4B as the lower end and the area of a lung field having the lower edge in FIG. 4B as the lower end. Let one of the areas be S_LAT_A and the other thereof be S_LAT_B.
(2) Add the areas of these lung fields together and divide the result by two, thereby obtaining S_LAT

S_LAT=(S_LAT_A+S_LAT_B)/2 (Equation 2)

In radiographing, the longer the distance between the examinee M and the radiation detector 13 is, the larger the subject is imaged. In a frontal chest radiograph, the distance between the radiation detector 13 and the left lung and the distance between the radiation detector 13 and the right lung are the same. Meanwhile, in a lateral chest radiograph, the lung field far from the radiation detector 13 is imaged with a larger ratio of itself to the lung field near to the radiation detector 13 than the actual ratio. Hence, when the left lung and the right lung are distinguishable from one another in the lateral chest dynamic image, it is preferable, in the lateral chest dynamic image, to identify the lung field far from the radiation detector 13, and correct the area of the identified lung field. The area may be corrected by the following (1) to (5).

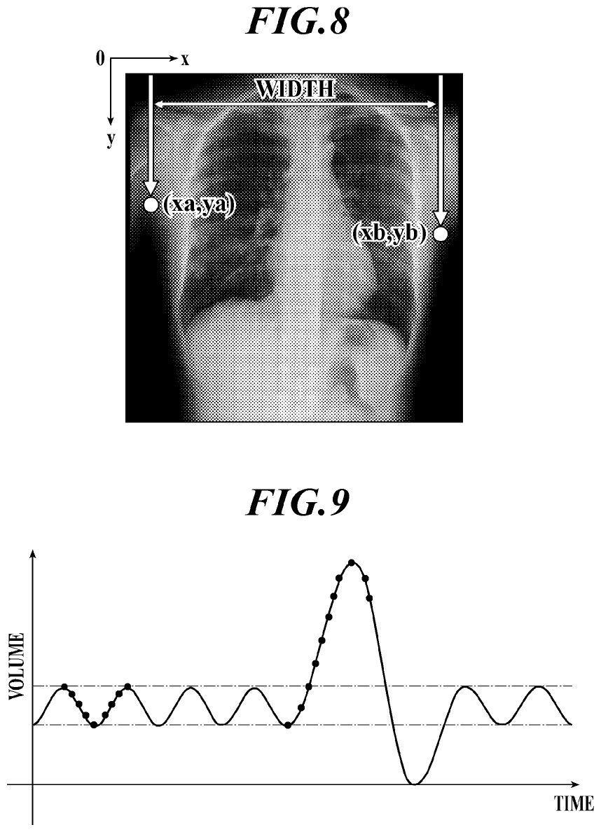

(1) In each lateral chest frame image, identify the lung field positioned far from the radiation detector 13 in the dynamic imaging from examination information and so forth attached to the lateral chest dynamic image. In this embodiment, the lung field positioned far from the radiation detector 13 in the dynamic imaging is the left lung field.
(2) Obtain the area S_LAT_R of the right lung field and the area S_LAT_L of the left lung field from the lateral chest frame image.
(3) Calculate the width of shoulders from the frontal chest frame image. For example, as shown in FIG. 8, scan the frontal chest frame image from the upper end downward such that the signal value changes from a signal value of a direct exposure part to a signal value of the subject, and then the signal value of the direct exposure part again, thereby reaching a point on the right side and a point on the left side each having the signal value of the direct exposure part and having the smallest value on y axis, wherein the signal values of the direct exposure part and the subject are distinguishable from one another with a preset threshold value; obtain (xa, ya) and (xb, yb) of the points; and calculate (xb−xa) as the width of shoulders Width.
(4) Correct the area S_LAT_L of the left lung field by Equation 3 below to obtain the corrected area S_LAT_L_{corrected} of the left lung field, wherein the distance between the tube of the radiation source 11 and the left lung field is expressed by (SID−Width), and an enlargement ratio of the left lung field is expressed by SID/(SID−Width).

S_LAT_L_{corrected}=S_LAT_L×(SID−Width)/SID (Equation 3)

(5) Obtain the area S_LAT of the lateral lung field by Equation 4 below.

S_LAT=(S_LAT_R+S_LAT_L_{corrected})/2 (Equation 4)

The above can accurately estimate the volume of the lung fields, and accordingly accurately estimate respiratory function indexes.

An atelectasis part(s) is a part where alveoli are collapsed, and does not fulfill the respiratory function. In order to estimate the respiratory function, the volume of the atelectasis part is excluded from the volume of the lung fields. It is preferable, when calculating the areas of the lung fields from the frontal chest frame image and the lateral chest frame image, to calculate the areas with the area(s) of the atelectasis part(s) excluded. The atelectasis part may be excluded by the following (1) to (3).

(1) Extract, as the atelectasis part, a region(s) having a signal value lower than a predetermined threshold value in the lung fields because the atelectasis part has a lower signal value (density value) than its surroundings.
(2) Calculate the area of the extracted atelectasis part.
(3) Subtract the area of the atelectasis part from the area (S_PA_R, S_PA_L, S_LAT) of the lung field.

This can estimate respiratory function indexes by using only part of the lung fields fulfilling the respiratory function, and more accurately estimate the respiratory function indexes.

Next, the controller 31 corrects the calculated areas of the lung fields on the basis of set values of the imaging conditions in the dynamic imaging, the imaging conditions affecting the area and the volume of the subject that are calculated from frame images of dynamic images (Step S15).

In Step S15, for example, the controller 31 refers to the storage 32, and reads the imaging conditions, which affect the area and the volume that are calculated on the basis of images obtained by imaging, and their reference values and correction coefficients. Examples of the imaging conditions, which affect the area and the volume that are calculated on the basis of images obtained by imaging, include SID and the distance between an examinee M (subject) and the radiation detector 13. The controller 31 also obtains the set values of the respective imaging conditions in the dynamic imaging, which has been performed for the current test (examination). The set values of the imaging conditions in the dynamic imaging can be obtained, for example, from the information (radiation emission conditions, image reading conditions, examination information, etc.) attached to the dynamic images. As to the distance between the examinee M and the radiation detector 13, for example, the distance (space) between an imaging table for imaging in the decubitus position and the radiation detector 13 is stored in the storage 32 in advance, and the controller 31 reads and obtains the value of the distance stored in the storage 32 when the dynamic imaging performed is dynamic imaging in the decubitus position. It is noted that correction based on this imaging condition is not performed when the dynamic imaging performed is dynamic imaging in the standing position. The controller 31 corrects the areas of the lung fields on the basis of the obtained reference values and correction coefficients of the imaging conditions and the obtained set values of the imaging conditions in the dynamic imaging.

Different values of SID change the size of the subject in radiographs (the shorter the SID is, the larger the subject is imaged). Even when the same subject (chest) is imaged, the areas of the lung fields that are calculated from the image differ depending on SID. This affects the volume that is estimated on the basis on frame images of dynamic images. Hence, the controller 31 corrects the area S_PA_R of the right lung, the area S PAL of the left lung, and the area S_LAT of the lateral lung field by Equation 5 below using a reference value BASE_{SID} and a correction coefficient α_{SID} of SID. In Equation 5, SID represents a set value (m) of SID in the dynamic imaging, S represents the area (S_PA_R, S_PA_L, S_LAT) calculated from the frontal chest frame image or the lateral chest frame image, and S_{corrected} represents the corrected area (S_PA_R, S_PA_L, S_LAT).

S_{corrected}=S×SID/BASE_{SID}×α_{SID} (Equation 5)

Different values of the distance between the examinee M and the radiation detector 13 change the size of the subject in radiographs (the longer the distance is, the larger the subject is imaged). Even when the same subject (chest) is imaged, the areas of the lung fields that are calculated from the image differ depending on the above distance. This affects the volume that is estimated on the basis of frame images of dynamic images. When the subject is imaged by being placed on an imaging table for imaging in the standing position, the examinee M (subject) is brought into contact with the radiation detector 13. When the subject is imaged by being placed on the imaging table for imaging in the decubitus position, the radiation detector 13 is mounted on the lower side of the imaging table, so that a space is generated between the examinee M (subject) and the radiation detector 13. Hence, the controller 31 corrects the area SPAR of the right lung, the area S_PA_L of the left lung, and the area S_LAT of the lateral lung field by Equation 6 below using a reference value BASEₛᵤₚᵢₙₑ and a correction coefficient αₛᵤₚᵢₙₑ of the distance between the examinee M and the radiation detector 13. In Equation 6, D represents a set value (μm) of the distance between the examinee M and the radiation detector 13 in the dynamic imaging, S represents the area (S_PA_R, S_PA_L, S_LAT) calculated from the frontal chest frame image or the lateral chest frame image, and S_{corrected} represents the corrected area (S_PA_R, S_PA_L, S_LAT).

S_{corrected}=S×BASEₛᵤₚᵢₙₑ/D×αₛᵤₚᵢₙₑ (Equation 6)

Next, the controller 31 estimates, for each extracted set of the frontal chest frame image and the lateral chest frame image, the volume of the lung fields on the basis of the calculated (corrected) areas of the lung fields (Step S16).

The volume of the lung fields can be estimated by Equations (7) and (8) below as disclosed, for example, in Non-Patent Document 1.

Volume of Lung Fields to be Calculated Simply from X-ray Images=Volume of Right Lung+Volume of Left Lung=(S_PA_R×S_LAT)\hat{ }(¾)+(S_PAL×S_LAT)\hat{ }(¾) (Equation 7)

Estimated Value of Volume of Lung Fields=0.67×(Volume of Lung Fields to be Calculated Simply from X-ray Images)+160 [ml] (Equation 8)

When the imaging conditions of the frontal chest dynamic image and the lateral chest dynamic image are the same, the controller 31 may correct the volume of the lung fields calculated by Equation 7 without correcting the areas of the lung fields on the basis of the set values of the imaging conditions in the dynamic imaging, which is performed in Step S15. For example, when the set value of SID in the dynamic imaging differs from its reference value, the volume calculated by Equation 7 can be corrected by multiplying the volume by (SID/BASEₛᵤₚᵢₙₑ/D×β_{SID})\hat{ }(¾) using β_{SID} as its correction coefficient. When the set value of the distance between the examinee M and the radiation detector 13 in the dynamic imaging is different from its reference value, the volume calculated by Equation 7 can be corrected by multiplying the volume by (BASEₛᵤₚᵢₙₑ/D×βₛᵤₚᵢₙₑ)\hat{ }(¾) using βₛᵤₚᵢₙₑ as its correction coefficient.

Next, the controller 31 estimates the respiratory function indexes on the basis of the estimated volume of the lung fields (Step S17).

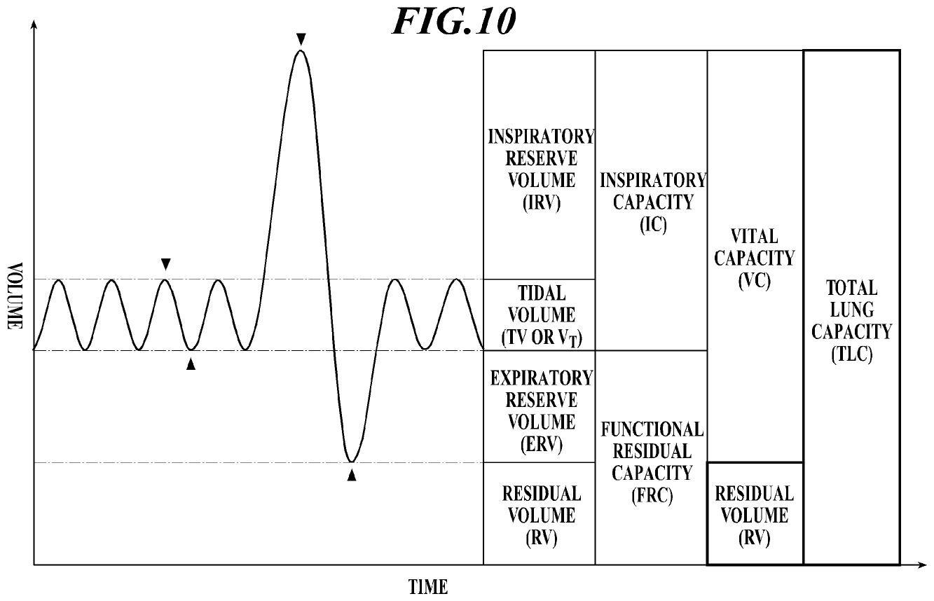

For example, as shown in FIG. 9, the controller 31 generates a waveform showing temporal change of the volume of the lung fields by plotting the volume of the lung fields, which has been estimated in Step S16 from each extracted set of the frontal chest frame image and the lateral chest frame image, and performing interpolation along the time axis, and estimates the generated waveform as a lung volume curve. The controller 31 can estimate, on the basis of values of points (peaks) indicated by triangles on the lung volume curve as shown in FIG. 10, respiratory function indexes unmeasurable with a spirometer, such as TLC and RV, and respiratory function indexes obtainable with a respiratory function test with a spirometer, such as IRV (Inspiratory Reserve Volume), IC (Inspiratory Capacity) and VC (Vital Capacity). For example, TLC can be estimated as the volume of the lungs at the forced maximal inspiratory position (maximal inspiratory position during deep breathing), and RV can be estimated as the volume of the lungs at the forced maximal expiratory position (maximal expiratory position during deep breathing).

Respiratory function indexes that can be estimated differ depending on whether the taken dynamic images include quiet breathing, deep breathing, or both. For example, TLC and RV can be estimated from images of deep breathing, whereas FRC (Functional Residual Capacity) can be estimated from images of quiet breathing. Hence, types of respiratory function index (respiratory function indexes) to be estimated are determined on the basis of the respiratory state during dynamic imaging. The respiratory state during dynamic imaging can be identified on the basis of, for example, the information attached to the dynamic images. Alternatively, the respiratory state during dynamic imaging may be determined by analyzing the lung volume curve generated from the dynamic images.

The respiratory state during dynamic imaging can be automatically determined by the following algorithm (1) to (4). Hereinafter, the algorithm for automatically determining the respiratory state during dynamic imaging will be described with reference to FIG. 11.

Instead of the lung volume curve, a waveform showing temporal change of a representative signal value of the entire image, the position of the diaphragm or the area(s) of the lung field(s) obtained from the frontal chest dynamic image and/or the lateral chest dynamic image may be used.

(1) Obtain the maximum value Max and the minimum value Min of the lung volume curve.
(2) Calculate the median Med=(Max+Min)/2, and draw a reference line on the lung volume curve passing a point M of the median Med.
(3) Make a determination on each combination of Maxₙ and Minₙ (n=1, 2, 3, . . . ) with the following conditions, wherein Maxₙ represents an extreme value in the +direction (peak of a convex upward) from the reference line, and Minₙ represents an extreme value in the −direction (peak of a convex downward) from the reference line.
(3-1) When Maxₙ−Minₙ>Thₜ and Maxₙ−Minₙ<Th_{d}, determine that quiet breathing Flagₜ=1 (true), and determine that deep breathing Flag_{d}=0 (false) and noise Flagₙ=0 (false).
(3-2) When Maxₙ−Minₙ>Th_{d}, determine that Flag_{d}=1, and determine that Flagₜ=0 and Flagₙ=0.
(3-3) Other than the above, determine that Flagₙ=1, and determine that Flagₜ=0 and Flag_{d}=0.

In the above, Thₜ (threshold value for quiet breathing)<<Th_{d} (threshold value for deep breathing), and each Flag is Bool type.

(4) Take OR of Flag when all the extreme values have been retrieved, and when it is 1, determine that quiet breathing is included. Take OR of Flag_{d} when all the extreme values have been retrieved, and when it is 1, determine that deep breathing is included. When both Flagₜ and Flag_{d} are 1, determine that quiet breathing and deep breathing are included. A section of frame images where Flagₜ=1 can be determined as a section of quiet breathing, and a section of frame images where Flag_{d}=1 can be determined as a section of deep breathing.

The above algorithm (1) to (4) for determining the respiratory state can automatically determine which frame image (frame number) comes under quiet breathing (end expiratory position, end inspiratory position) and which frame image (frame number) comes under deep breathing (forced maximal expiratory position, forced maximal inspiratory position) when both quiet breathing and deep breathing are included as shown in FIG. 11, and estimate respiratory function indexes.

As shown in FIG. 11, when a plurality of respiratory phases that are used for estimating a respiratory function index(es) (e.g. phases of any of the end expiratory position, end inspiratory position, forced maximal expiratory position and forced maximal inspiratory position) are included in the dynamic images, it is preferable, for estimating the respiratory function index(es), to use the respiratory phase in the last cycle, where breathing is stable. Alternatively, a representative value (e.g. the maximum value, minimum value, mean value, etc.) of peaks of cycles may be calculated and used for estimating the respiratory function index(es). Still alternatively, the lung volume curve or a waveform showing temporal change of the characteristic amount calculated in Step S12 may be displayed on the display 34, and a respiratory phase specified by the user operating the operation unit 33 may be used therefor. When the waveform showing the characteristic amount is displayed, waveforms showing the characteristic amount calculated from the frontal chest dynamic image and the lateral chest dynamic image may be displayed next to one another (e.g. arranged vertically). For the user to readily understand correspondence of phases that match, the waveforms may be shifted such that a reference point (e.g. forced maximal inspiratory position) of one waveform coincides with that of the other waveform.

VC is a respiratory function index measurable with a spirometer. Hence, the following may be performed: obtain the actual measurement value of VC with a spirometer (obtain, from a spirometer, an electronic health record system or the like, the actual measurement value of VC that is input with the operation unit 33 or received through the communication unit 35); calculate a correction coefficient C on the basis of the obtained VC_{Real} and the VC (VC_{Im}) calculated from the dynamic images; and on the basis of the correction coefficient C, correct the value of another type of respiratory function index (e.g. TLC or RV) calculated from the dynamic images. The correction coefficient C can be obtained by Equation 9 below.

C=VC_{Real}/NC_{Im} (Equation 9)

Thus, combining dynamic images with a test with a spirometer can calculate respiratory function indexes unmeasurable with a spirometer, such as TLC and RV, with a high degree of accuracy, without carrying out any detailed pulmonary function test that is expensive and casts a heavy burden on patients, such as body plethysmography.

When the calculation result of Equation 9 is outside a predetermined range, it is preferable to display an alert or output an alert with audio.

Then, the controller 31 causes the display 34 to display (the estimated values of) the estimated respiratory function indexes (Step S18), and ends the respiratory function index estimation process. The controller 31 may cause the display 34 to display the frame images of each set extracted in Step S13 next to one another together with (the estimated values of) the respiratory function indexes.

Hereinafter, examples of how the display 34 displays the estimation results of the respiratory function indexes in Step S18 will be described.

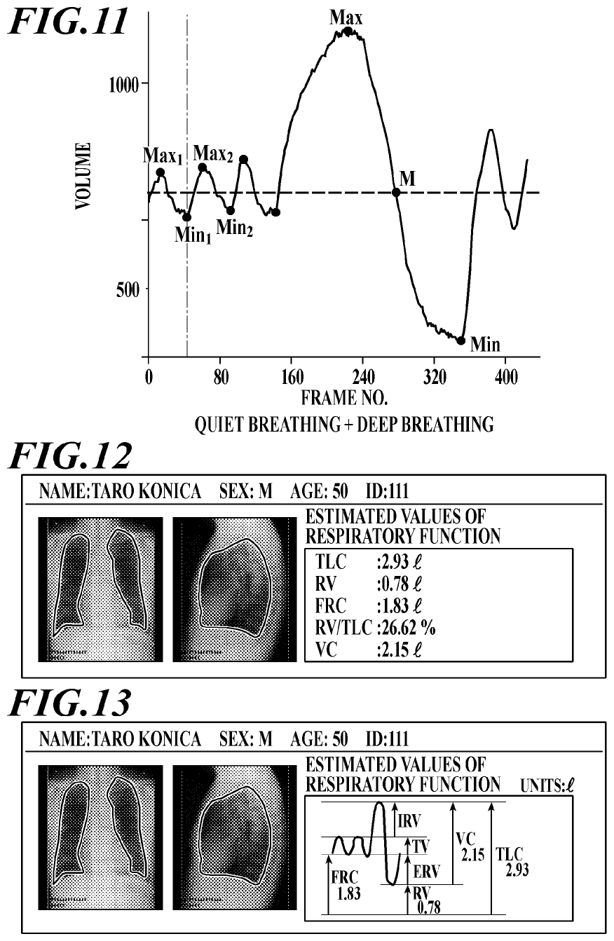

For example, as shown in FIG. 12, representative frame images (e.g. images at the forced maximal inspiratory position) of the frontal chest dynamic image and the lateral chest dynamic image are displayed, and also the estimated values of the respiratory function indexes are displayed as they are, namely in numerical values.

Alternatively, as shown in FIG. 13, representative frame images (e.g. images at the forced maximal inspiratory position) of the frontal chest dynamic image and the lateral chest dynamic image are displayed, and also the estimated values of the respiratory function indexes are displayed on a graph of lung capacity. This allows the user to intuitively grasp the estimated values of the respiratory function indexes.

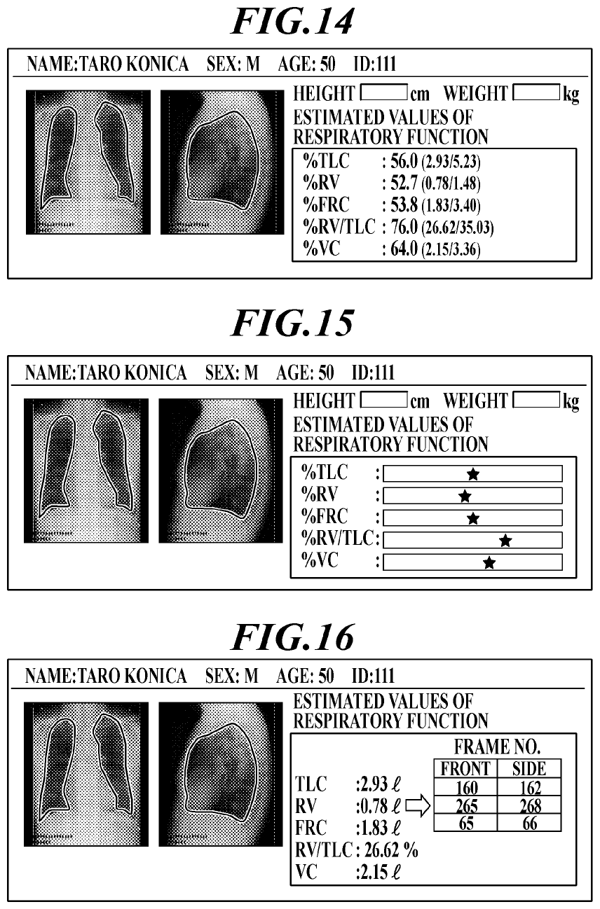

Alternatively, as shown in FIG. 14, representative frame images (e.g. images at the forced maximal inspiratory position) of the frontal chest dynamic image and the lateral chest dynamic image are displayed, and also ratios (%) of the estimated values of the respiratory function indexes to their respective predictive values calculated from the sex, height, age, weight and so forth of the patient are displayed. Also, by using parentheses, “(estimated value/predictive value)” is displayed together with each of the ratios (%). This can display the estimation results of the respiratory function indexes in a style highly affinitive with medical treatment. Information necessary for calculation of the predictive values, such as the sex, height, age and weight of the patient, may be obtained from the information attached to the dynamic images, or may be input on the screen.

The predictive values of the respiratory function indexes may be calculated using known calculation formulae for calculating predictive values of respiratory function indexes stored in the storage 32, or may be calculated using calculation formulae calculated by machine learning or the like from a large amount of data in each of which information such as sex, height, age and weight is associated with the actual measurement values of respiratory function indexes. Examples of the known calculation formulae that can be used include: Baldwin's formula (VC-B formula), Berglund's formula (FEV₁-B formula), VC prediction formula (VC-J formula) and FEV1 prediction formula (FEV₁-J formula) disclosed in Reference Document 4 (Mie Aoki, Shinobu Osanai, Toshiyuki Ogasa, Noriyoshi Yamazaki, Kensuke Ishida, Hiroaki Nakata, Shoko Nakao, Eri Toyoshima, Naoyuki Hasebe and Yoshinobu Ohsaki, “Comparison between predicted equations obtained by standard Japanese values and present predicted equations for vital capacity and forced expiratory volume in one second”, The Journal of the Japanese Respiratory Society, 48(5), 2010); and prediction formulae of TLC, RV and FRC using sex, height and age disclosed in Reference Document 5 (J. Stocks, Ph. H. Quanjer, “REFERENCE VALUES FOR RESIDUAL VOLUME, FUNCTIONAL RESIDUAL CAPACITY AND TOTAL LUNG CAPACITY”, Eur Respir J, 1995, 8, P.492-P.506).

Alternatively, as shown in FIG. 15, representative frame images (e.g. images at the forced maximal inspiratory position) of the frontal chest dynamic image and the lateral chest dynamic image are displayed, and also ratios (%) of the estimated values of the respiratory function indexes to their respective predictive values calculated from the sex, height, age, weight and so forth of the patient are displayed on scale bars (or on a radar chart). This allows the user to intuitively and readily grasp the ratios (%) of the estimated values of the respiratory function indexes to their respective predictive values calculated from the sex, height, age, weight and so forth of the patient.

Alternatively, as shown in FIG. 16, representative frame images (e.g. images at the forced maximal inspiratory position) of the frontal chest dynamic image and the lateral chest dynamic image are displayed, and also the estimated values of the respiratory function indexes are displayed as they are, namely in numerical values, together with the frame numbers of frame images of the frontal chest dynamic image and the lateral chest dynamic image used for the estimation. This allows the user to easily check the frame images that are the grounds for the estimated values of the respiratory function indexes.

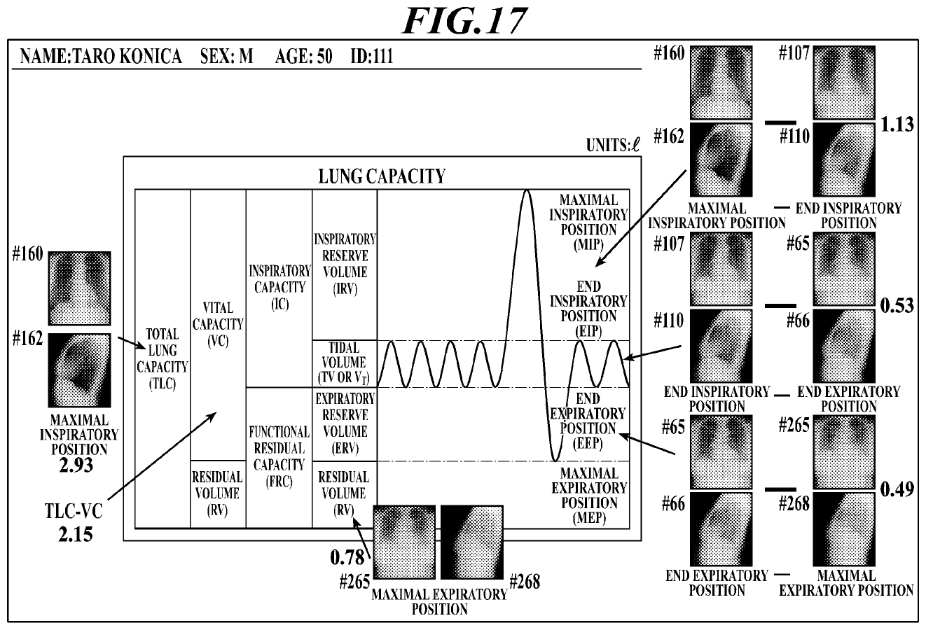

Alternatively, as shown in FIG. 17, in connection with a graph of lung capacity, the estimated values of the respiratory function indexes are displayed together with thumbnail images and the frame numbers (with “#” in FIG. 17) of frame images of the frontal chest dynamic image and the lateral chest dynamic image used for the estimation so as to be associated with one another. This allows the user to readily check the frame images that are the grounds for the estimated value of the respiratory function indexes. Instead of displaying information on all the calculated respiratory function indexes as shown in FIG. 17, information on a respiratory function index corresponding to a point on the graph of lung capacity pressed with the operation unit 33 may be displayed, for example.

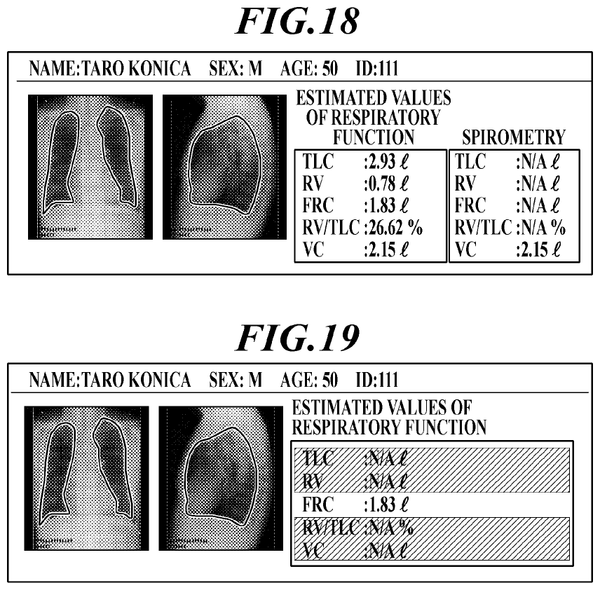

Alternatively, as shown in FIG. 18, representative frame images (e.g. images at the forced maximal inspiratory position) of the frontal chest dynamic image and the lateral chest dynamic image are displayed, and also the estimated values of the respiratory function indexes are displayed together with results (actual measurement values) of a test with a spirometer (spirometry). This allows the user to readily refer to the actual measurement values of the spirometry.

As described above, types of respiratory function index (respiratory function indexes) that can be estimated differ depending on the respiratory state during dynamic imaging Hence, as shown in FIG. 19, indexes that have not been estimated due to the respiratory state during dynamic imaging may be displayed by being masked. This allows the user to recognize that the masked indexes are indexes that have not been estimated due to the respiratory state during dynamic imaging.

When dynamic imaging of the front of the chest is performed under the quiet breathing state and also performed under the deep breathing state, and further dynamic imaging of a side of the chest is performed under the quiet breathing state and also performed under the deep breathing state, frontal chest dynamic images taken under the quiet breathing state and the deep breathing state and lateral chest dynamic images taken under the quiet breathing state and the deep breathing state are present. Then, the controller 31 determines whether each dynamic image is a dynamic image of quiet breathing or a dynamic image of deep breathing by referring to the information attached to each dynamic image or by carrying out, for each dynamic imaging (dynamic image), the above-described algorithm for automatically determining the respiratory state, and performs the respiratory function index estimation process using the dynamic images taken from different imaging directions (front and side) under the same respiratory state. This can prevent respiratory function indexes from being estimated erroneously using a frontal chest dynamic image and a lateral chest dynamic image taken from different imaging directions under different respiratory states.

As described above, the controller 31 of the diagnostic console 3: extracts at least one set of frame images having the values of the characteristic amount most similar to one another, the characteristic amount relating to the dynamic state of the lung fields due to the respiration, from a frontal chest dynamic image and a lateral chest dynamic image; on the basis of the areas of the lung fields calculated from the frame images, estimates the volume of the lung fields for each of the extracted at least one set; and on the basis of the estimated volume, estimates a respiratory function index(es). This can estimate the volume of the lung fields from frame images of a frontal chest dynamic image and a lateral chest dynamic image, the frame images having the phases of the dynamic state of the lung fields most similar to one another and showing the lung fields substantially matching in size, and accordingly improve estimation accuracy of the volume of the lung fields and improve estimation accuracy of respiratory function indexes. Also, this can estimate, other than TLC and so forth, a lung volume curve and respiratory function indexes that are obtained by a respiratory function test(s) (with a spirometer).

Further, the controller 31: corrects, on the basis of a set value(s) of an imaging condition(s) in radiographing the dynamic state, the imaging condition affecting the area and the volume of a subject that are calculated from dynamic images of the subject, (i) the areas of the lung fields calculated from the frame images of the frontal chest dynamic image and the lateral chest dynamic image for estimating the volume of the lung fields or (ii) the volume of the lung fields; and estimates, on the basis of (i) the volume calculated on the basis of the corrected areas or (ii) the corrected volume, the respiratory function index of the lung fields. This can prevent the volume from being estimated (calculated) inaccurately or the estimated (calculated) volume from being inaccurate due to set values of imaging conditions in dynamic imaging performed, and accordingly improve estimation accuracy of the volume of the lung fields and improve estimation accuracy of respiratory function indexes.

Further, the controller 31 obtains the actual measurement value of a respiratory function index (type of respiratory function index) obtained by a different test, such as spirometry, and on the basis of the obtained actual measurement value and an estimation result of a respiratory function index (type) identical with the respiratory function index (type) of the actual measurement value, corrects an estimation result of another respiratory function index (type). This can estimate respiratory function indexes unmeasurable with a spirometer, such as TLC and RV, with a high degree of accuracy, without carrying out any detailed pulmonary function test that is expensive and casts a heavy burden on patients, such as body plethysmography.

Further, the controller 31 causes the display 34 to display the estimated respiratory function index (estimation result). This allows the user to check estimated respiratory function indexes (estimated values).

Further, the controller 31 causes the display 34 to display the frame images of the at least one set extracted from the dynamic images next to one another. This allows the user to check dynamic images used for estimating respiratory function indexes.

Those described in the above embodiment are preferred examples of the present invention, and not intended to limit the present invention.

For example, in the above embodiment, the lung-apex-to-diaphragm distance is used as the characteristic amount relating to the dynamic state of the lung fields. However, this is not intended to limit the present invention. When a point on a structure that can be regarded as hardly changing its position by respiration and a point on a structure that changes its position according to respiratory phases can be specified in both a frontal chest dynamic image and a lateral chest dynamic image, use of a distance between these points can produce the same effects as the above embodiment. Examples of the distance that can be used as the characteristic amount include: a distance between the aortic arch and the apex of the diaphragm; and a distance between the upper end of the third thoracic vertebra and the apex of the diaphragm. The characteristic amount is not even limited to distances. Examples of the characteristic amount other than distances include: the area(s) of the lung field(s); and signal values (density values) in the lung field(s) (e.g. a representative value, such as the mean value, maximum value, minimum value, etc.). As described above, these change according to the dynamic state of the lung fields due to the respiration, and are types of the characteristic amount having an extremely high correlation with change of the size of the lung fields with the respiration. The area(s) of the lung field(s) and the density value(s) in the lung field(s) have different absolute values between a frame image of a frontal chest dynamic image and a frame image of a lateral chest dynamic image even when these frame images have the same phase. Hence, when either of these is used as the characteristic amount, it is used after normalized with its maximum value and minimum value, for example.

Further, for example, in the above embodiment, as a method for obtaining the volume of the lung fields using a frontal chest dynamic image and a lateral chest dynamic image, the method of obtaining the volume of the lung fields from the areas of the lung fields calculated from frame images of a frontal chest dynamic image and a lateral chest dynamic image is used. However, the method for obtaining the volume of the lung fields using a frontal chest dynamic image and a lateral chest dynamic image is not limited thereto.

For example, as disclosed in Reference 3 and Reference 6 (R J Pierce et al. “Estimation of lung volumes from chest radiographs using shape information”, Thorax 1979 34: 726-734), the volume of lung fields may be obtained on the basis of knowledge that the cross-sectional shapes of the lung fields are elliptical, and the lung fields are expressed as a cylinder(s) constituted of a series of ellipses. For example, the volume of the lung fields may be obtained as follows: identify regions of the lung fields (thorax), heart, spine and so forth in frame images of a frontal chest dynamic image and a lateral chest dynamic image; align the frame images in the same vertical plane; divide the frame images into a large number of horizontal slices; obtain the diameters of the lung fields and the structures (e.g. heart, spine, etc.) inside the lung fields in the slices (widths of regions of the lung fields and the structures in the frame image of the frontal chest dynamic image) and the thicknesses of the lung fields and the structures (e.g. heart, spine, etc.) inside the lung fields in the slices (widths of regions of the lung fields and the structures in the frame image of the lateral chest dynamic image); estimate the areas of the cross-sectional regions (ellipses) of the lung fields and the structures in each slice; subtract the areas of the cross-sectional regions of the structures from the area(s) of the cross-sectional region(s) of the lung fields (thorax) in each slice; and sum information from all of the slices. It is preferable to correct the area(s) and the volume calculated or to be calculated by this method too on the basis of the set values of the imaging conditions, which affect the area and the volume that are calculated on the basis of images obtained by imaging.

In the above method for estimating the volume of the lung fields on the basis of the knowledge that the cross-sectional shapes of the lung fields are elliptical (hereinafter “ellipse-based volume estimation method”), it is necessary to extract the regions of the structures, such as the heart, from the frame images of the frontal chest dynamic image and the lateral chest dynamic image. Because the heart changes its size with the heartbeat, it is preferable, as to the heart too (in addition to the lung fields), to extract a set of frame images having the phases most similar to one another in cardiac cycles from the frontal chest dynamic image and the lateral chest dynamic image, and obtain the volume of the heart on the basis of the extracted set of the frame images.

Further, in the above embodiment, the present invention is applied to the case where respiratory function indexes are estimated from a frontal chest dynamic image and a lateral chest dynamic image. However, the present invention is also applicable to a case where indexes for evaluating a cardiac function (cardiac function indexes) are estimated. For example, the controller 31 may estimate a cardiac function index(es) as follows: identify contours of the heart in each frame image of each of a frontal chest dynamic image and a lateral chest dynamic image; calculate the characteristic amount relating to the dynamic state of the heart; extract at least one set of frame images having the calculated values of the characteristic amount most similar to one another around the end of a diastole and/or the end of a systole; calculate the area of the heart from each of the frame images extracted from the frontal chest dynamic image and the lateral chest dynamic image; estimate the volume of the heart on the basis of the area calculated from each of the frame images extracted from the frontal chest dynamic image and the lateral chest dynamic image; and estimate a cardiac function index(es) on the basis of the estimated volume. Examples of types of cardiac function index (cardiac function indexes) include the volume of the heart at the end of a diastole and the volume of the heart at the end of a systole. As with the above embodiment, it is preferable to: correct the area and the volume of the heart calculated or to be calculated from the dynamic images for calculating the cardiac function index(es) on the basis of the set value(s) of the imaging condition(s) in radiographing the dynamic state, the imaging condition(s) affecting the area and the volume that are calculated on the basis of images obtained by imaging; and calculate the cardiac function index(es) on the basis of the volume calculated on the basis of the corrected area or the corrected volume.

The contours of the heart may be identified on the basis of contours of the heart that are manually specified with user operations, or may be automatically identified using known image processing, such as edge detection, dynamic contour model or region segmentation. As a method for estimating the volume of the heart, the ellipse-based volume estimation method disclosed in Reference Document 4 may be used, for example.

Examples of the characteristic amount relating to the dynamic state of the heart include: the area and the width of a heart region; and a high-frequency component of density (e.g. a representative value, such as the mean value, maximum value, minimum value, etc.) in the heart region. These change according to the dynamic state of the heart due to the heartbeat, and are types of the characteristic amount having an extremely high correlation with change of the size of the heart with the heartbeat. The area and the width of the heart region and the value(s) of the high frequency component in the heart region have different absolute values between a frame image of a frontal chest dynamic image and a frame image of a lateral chest dynamic image even when these frame images have been taken at the same phase in cardiac cycles. Hence, when any of these is used as the characteristic amount, it is used after normalized with its maximum value and minimum value, for example.

Whether or not the frame images are frame images around the end of a diastole or the end of a systole can be determined on the basis of, for example, whether or not the values of the characteristic amount calculated from the frame images are within a preset range.

Further, in Step S13 of the above embodiment, multiple sets of frame images are extracted from a frontal chest dynamic image and a lateral chest dynamic image. However, only one set of frame images may be extracted therefrom.

For example, when only TLC is obtained as a respiratory function index, a set of frame images having the calculated values of the characteristic amount most similar to one another around the forced maximal inspiratory position (the maximal inspiratory position during deep breathing) is extracted from the frontal chest dynamic image and the lateral chest dynamic image, and the volume of the lung fields is obtained on the basis of the extracted frame images, so that TLC is estimated. Further, for example, when only RV is obtained as a respiratory function index, a set of frame images having the calculated values of the characteristic amount most similar to one another around the forced maximal expiratory position (the maximal expiratory position during deep breathing) is extracted from the frontal chest dynamic image and the lateral chest dynamic image, and the volume of the lung fields is obtained on the basis of the extracted frame images, so that RV is estimated. Either of these can shorten processing time. Whether or not the frame images are frame images around a predetermined respiratory phase can be determined on the basis of, for example, whether or not the values of the characteristic amount calculated from the frame images are within a preset range.

Further, in the above embodiment, the volume of the subject is estimated using dynamic images thereof taken from two different directions, namely using a frontal chest dynamic image and a lateral chest dynamic image. However, the imaging direction is not limited to the front and a side of the subject, and the volume of the subject may be estimated using dynamic images thereof taken from other multiple directions.

Further, in the above embodiment, the present invention is applied to dynamic images of the lung fields or the heart as the subject. However, the present invention may be applied to dynamic images of another part, such as a joint of a limb, as the subject.

Further, in the above embodiment, a hard disk, a nonvolatile semiconductor memory or the like is used as a computer readable medium of the programs of the present invention. However, this is not a limitation. As the computer readable medium, a portable storage medium, such as a CD-ROM, can also be used. Further, as a medium to provide data of the programs of the present invention, a carrier wave can be used.

In addition to the above, detailed configuration and detailed operation of each apparatus or the like of the dynamic image analysis system can also be appropriately modified without departing from the scope of the present invention.

Although some embodiments of the present invention have been described and illustrated in detail, the disclosed embodiments are made for purposes of not limitation but illustration and example only. The scope of the present invention should be interpreted by terms of the appended claims

What is claimed is:

1. A dynamic image analysis apparatus comprising a hardware processor that:
from each frame image of each of dynamic images obtained by radiographing a cyclic dynamic state of a subject from different directions, calculates a characteristic amount relating to the dynamic state of the subject;
based on the calculated characteristic amount, extracts at least one set of frame images having phases of the dynamic state of the subject most similar to one another from the dynamic images;
from each of the frame images of each of the extracted at least one set, calculates an area of the subject;
based on the calculated area of the subject, calculates a volume of the subject for each of the extracted at least one set;
based on a set value of an imaging condition in the radiographing, the imaging condition affecting the area and the volume of the subject that are calculated from the dynamic images, corrects (i) the calculated area of the subject or (ii) the calculated volume of the subject; and
based on (i) the volume calculated based on the corrected area or (ii) the corrected volume, estimates an evaluation index of a function of the subject.

2. The dynamic image analysis apparatus according to claim 1, wherein the imaging condition includes at least one of a distance between a tube of a radiation source and a radiation detector during the radiographing and a distance between the subject and the radiation detector during the radiographing.

3. The dynamic image analysis apparatus according to claim 1, wherein the hardware processor:
estimates multiple types of the evaluation index, thereby obtaining evaluation results of the types of the evaluation index;
obtains an actual measurement value of a type of the evaluation index obtained by a different test; and
based on the obtained actual measurement value and, among the evaluation results of the types, an estimation result of a type identical with the type of the actual measurement value, corrects an estimation result of another type among the evaluation results of the types.

4. The dynamic image analysis apparatus according to claim 1,
wherein the dynamic images include a dynamic image of a front of a chest and a dynamic image of a side of the chest, and
wherein the evaluation index of the function of the subject includes an evaluation index of a respiratory function.

5. The dynamic image analysis apparatus according to claim 1, further comprising a display that displays the estimated evaluation index.

6. The dynamic image analysis apparatus according to claim 5, wherein the display further displays the frame images of the extracted at least one set next to one another.

7. A dynamic image analysis system comprising:
the dynamic image analysis apparatus according to claim 1;
an imaging apparatus that radiographs the dynamic state of the subject; and
a display apparatus that displays the estimated evaluation index.

8. A non-transitory computer-readable storage medium storing a program to cause a computer to:
from each frame image of each of dynamic images obtained by radiographing a cyclic dynamic state of a subject from different directions, calculate a characteristic amount relating to the dynamic state of the subject;
based on the calculated characteristic amount, extract at least one set of frame images having phases of the dynamic state of the subject most similar to one another from the dynamic images;
from each of the frame images of each of the extracted at least one set, calculate an area of the subject;
based on the calculated area of the subject, calculate a volume of the subject for each of the extracted at least one set;
based on a set value of an imaging condition in the radiographing, the imaging condition affecting the area and the volume of the subject that are calculated from the dynamic images, correct (i) the calculated area of the subject or (ii) the calculated volume of the subject; and
based on (i) the volume calculated based on the corrected area or (ii) the corrected volume, estimate an evaluation index of a function of the subject.
